(19) United States
(12) Patent Application Publication
(10) Pub. No.: US 2013/0233798 A1
(43) Pub. Date: Sep. 12, 2013

(54) BLOOD TREATMENT DEVICE

(71) Applicants: Christoph Wiktor, Gelnhausen (DE); Arne Peters, Bad Homburg (DE); Alexander Heide, Eppstein (DE)

(72) Inventors: Christoph Wiktor, Gelnhausen (DE); Arne Peters, Bad Homburg (DE); Alexander Heide, Eppstein (DE)

(73) Assignee: Fresenius Medical Care Deutschland GmbH, Bad Homburg (DE)

(21) Appl. No.: 13/597,517

(22) Filed: Aug. 29, 2012

Related U.S. Application Data
(60) Provisional application No. 61/530,554, filed on Sep. 2, 2011.

(30) Foreign Application Priority Data
Sep. 2, 2011 (DE) 10 2011 112 221.8

(51) Int. Cl. A61M 1/16

(52) U.S. Cl. CPC A61M 1/16
USPC 210/646; 210/194; 210/87; 210/137

(57) ABSTRACT

The present invention relates to a blood treatment device with at least one extra-corporeal blood circuit with a filter element and with at least one centrifugal pump means, wherein furthermore a monitoring means is provided, by means of which the flow resistance of the extracorporeal circuit can be determined, wherein the flow resistance can at least partly be determined with reference to the flow in at least one portion of the extracorporeal blood circuit and the speed of the centrifugal pump means. Furthermore, the present invention relates to a monitoring means for a blood treatment device and to a method for operating a blood treatment device.

The present invention relates to a blood treatment device with at least one impeller pump, a monitoring means for a blood treatment device, and a method for operating a blood treatment device.

Centrifugal pumps or impeller pumps are pressure sources and are employed in the field of blood delivery. Such centrifugal pumps or impeller pumps can also be used as an alternative to positive-displacement pumps, in particular peristaltic hose pumps.

In contrast to flow sources such as peristaltic hose pumps, centrifugal pumps or impeller pumps do not produce a speed-dependent flow rate, but a speed-dependent differential pressure. The flow rate of centrifugal pumps substantially results from the flow resistance of the circuit and the viscosity of the fluid.

In extracorporeal circuits, the flow resistance plays a great role: A changed resistance e.g. of a dialysis filter in an extracorporeal blood circuit for blood treatment indicates for example the clotting of blood. So-called kinking, clotting or stenoses can lead to a great change in the resistance of the lines. The suction of the arterial needle or the catheter to a vessel of the patient leads to a great increase of the flow resistance in the patient access. Under unfavorable conditions, all of the aforementioned events can occur relatively frequently during extracorporeal blood treatments and furthermore can potentially lead to a damage of the blood. Against this background it is important to detect such events as early as possible and initiate countermeasures.

From DE 10 2007 007 198 A1 there is already known a method and an apparatus for monitoring and optimizing a blood circuit effected by a pump. An optimization of the blood flow is achieved here for the automatic control of blood pumps by periodic speed interventions and the occurring flow changes via a formed differential quantity and a control algorithm. In addition, the location of possible flow resistances on the venous or arterial side can be determined.

Furthermore, in connection with the blood treatment cassette systems are known, in which at least a part of the extracorporeal blood circuit is combined in a disposable cassette. Such cassette systems are already disclosed for example by DE 10 2009 024 465 A1 and DE 10 2009 018 664 A1.

In view of the aforementioned background it would be desirable to be able to provide a simple and reliable flow resistance determination in blood treatment devices with extracorporeal circuits which include centrifugal pumps or impeller pumps.

Therefore, it is the object of the present invention to develop a blood treatment device, a monitoring means for a blood treatment device and a method for operating a blood treatment device as mentioned above in an advantageous way, in particular to the effect that a flow resistance determination becomes possible in a simple and reliable way with extracorporeal circuits driven by centrifugal pumps.

In accordance with the invention, this object is solved by a blood treatment device with the features of claim 1. Accordingly it is provided that a blood treatment device includes at least one extracorporeal blood circuit with a filter element and at least one centrifugal pump means, wherein furthermore a monitoring means is provided by means of which the flow resistance of the extracorporeal circuit can be determined, wherein the flow resistance can at least partly be determined with reference to the flow in at least one portion of the extracorporeal circuit and the speed of the centrifugal pump means.

The blood treatment device for example can be a hemodialysis machine for use in the hemodialysis, hemodiafiltration, hemofiltration etc.

The blood treatment device according to the invention in particular allows to determine a direct measure of the flow resistance of a circuit. In addition, it is possible that an initial test of parts of the extracorporeal blood circuit can be made. These parts in particular can be disposable articles.

In addition, it is possible for example that monitoring means of the extracorporeal blood circuit, such as flow sensors, can be subjected to an initial functional test.

In addition, it is possible that a flow resistance measurement can be performed independent of the viscosity of the medium delivered in the extracorporeal blood circuit, which is not the case for example in a pressure measurement. In flow resistance measurements based on pressure measurements it is required to determine the viscosity of the delivered medium, i.e. for example the viscosity of the blood.

Furthermore, it is possible that a continuous measurement can be performed during the blood flow control without an additional test.

Furthermore, it is very advantageously possible that the relation between speed and flow now can be used to determine the viscosity of the delivered medium, for example of the blood.

It can be provided that in a first step the flow in the at least one potion of the extra-corporeal blood circuit can be determined at a first speed by means of the monitoring means and in a second step the flow in the at least one portion of the extracorporeal blood circuit can be determined at a second speed, wherein by means of these values as a measure for the flow resistance the ratio of the differential values can be calculated. The measure for the flow resistance is the ratio of the differential values of the flow Q1 in the at least one portion of the extracorporeal blood circuit at a first speed n1 and the flow Q2 in the at least one portion of the extracorporeal blood circuit at a second speed n2, i.e. the value dQ/dn.

It is conceivable that by means of the monitoring means a faulty condition of the extracorporeal blood circuit can be detected when the measure for the flow resistance leaves a predefined range and/or exceeds or falls below a predefined threshold value.

Furthermore, it can be provided that by means of the monitoring means at least one countermeasure can be initiated upon detection of a faulty condition.

It is furthermore conceivable that the countermeasure comprises the triggering of an alarm and/or the changing of the speed of the centrifugal pump means and/or the transfer of the blood treatment device into a safe condition.

It is possible that at least one display means is provided, by means of which the measure for the flow resistance can be represented and/or displayed. This involves the advantage that an easily comprehensible non-technical representation of the flow resistance can be displayed for example on a user interface such as e.g. a display of the blood treatment device.

In addition, it can be provided that in the extracorporeal blood circuit at least one pressure detection means, in particular at least one pressure sensor is provided, by means of which the pressure in at least one portion of the extracorporeal blood circuit can be determined.

It is also conceivable that by means of the monitoring means a point in the extra-corporeal blood circuit with an increased flow resistance can be determined with reference to the pressure determined by means of the pressure detection means.

It is possible that the extracorporeal blood circuit is at least partly formed by a disposable cassette.

It is conceivable that the cassette includes at least one hose portion and/or at least one drip chamber and/or at least one valve and/or at least one filter and/or the filter element.

In addition, the present invention relates to a monitoring means with the features of claim 11. Accordingly, it is provided that a monitoring means is configured for a blood treatment device according to any of claims 1 to 10 with the monitoring means features according to any of claims 1 to 10.

Furthermore, the present invention relates to a method for operating a blood treatment device with the features of claim 12. Accordingly it is provided that in a method for operating a blood treatment device, in particular a blood treatment device according to any of claims 1 to 10, comprising at least one extracorporeal blood circuit with a filter element and with at least one centrifugal pump means, wherein furthermore a monitoring means is provided, by means of which the flow resistance of the extracorporeal circuit can be determined, the flow resistance is at least partly determined with reference to the flow in at least one portion of the extracorporeal circuit and the speed of the centrifugal pump means.

Furthermore, it is conceivable that in a first step the flow in the at least one portion of the extracorporeal blood circuit is determined at a first speed by means of the monitoring means and in a second step the flow in the at least one portion of the extracorporeal blood circuit is determined at a second speed, wherein by means of these values as a measure for the flow resistance the ratio of the differential values is calculated.

It is possible that by means of the monitoring means a faulty condition of the extra-corporeal blood circuit is detected, when the measure for the flow resistance leaves a predefined range and/or exceeds or falls below a predefined threshold value.

In addition, it can be provided that by means of the monitoring means at least one countermeasure is initiated upon detection of a faulty condition.

It is furthermore possible that the countermeasure comprises the triggering of an alarm and/or the changing of the speed of the centrifugal pump means and/or the transfer of the blood treatment device into a safe condition.

In addition, it can be provided that by means of the monitoring means a point in the extracorporeal blood circuit with an increased flow resistance can be determined with reference to a determined pressure in at least one first portion of the extracorporeal blood circuit or is determined in the presence of an increased flow resistance.

Further details and advantages of the invention will now be explained in detail with reference to an exemplary embodiment illustrated in the drawing.

In the drawing:

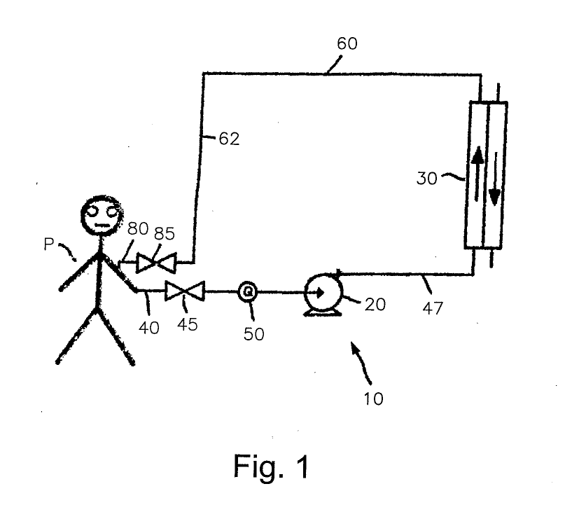

FIG. 1: shows a schematic representation of the extracorporeal circuit with centrifugal pump, dialyzer, flow sensor and safety clamps;

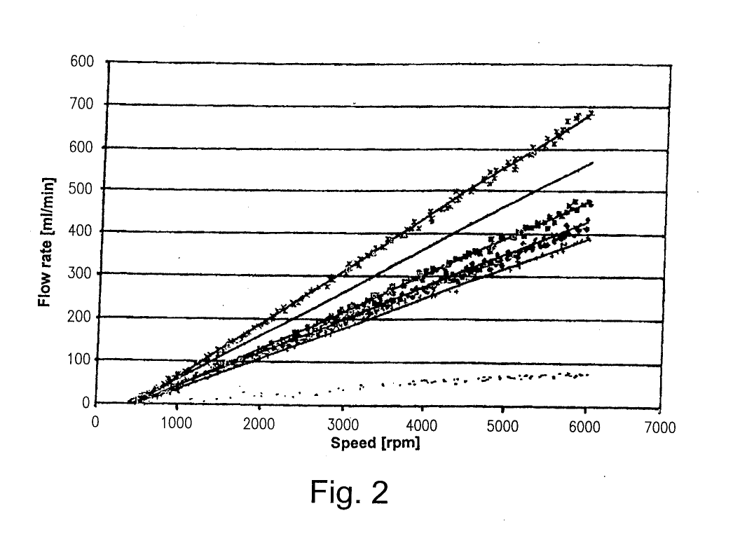

FIG. 2: shows a diagram concerning the flow rate in dependence on the speed at different flow resistances and constant viscosity;

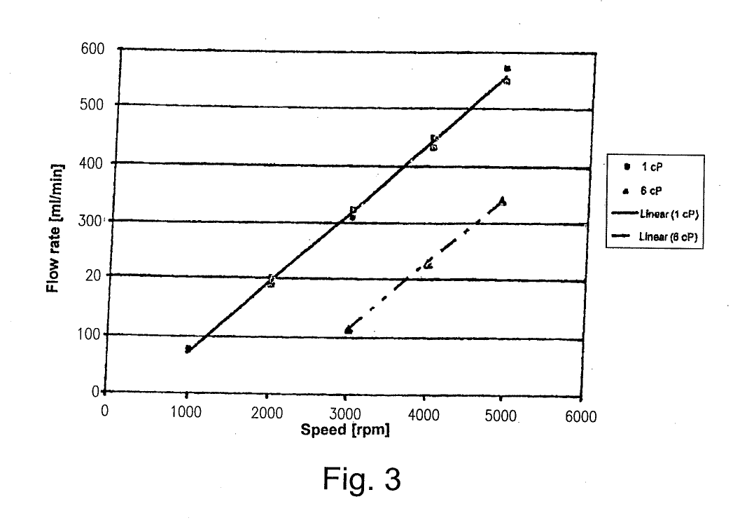

FIG. 3: shows a diagram concerning the flow rate in dependence on the speed at different viscosity and constant flow resistance;

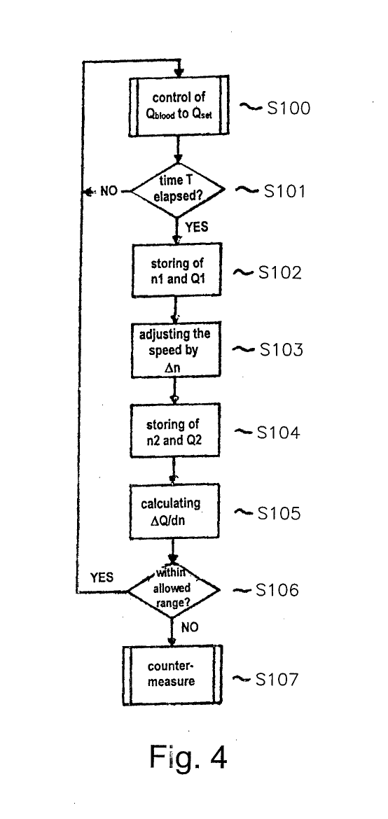

FIG. 4: shows a flow diagram of a reversible speed change for flow resistance determination; and

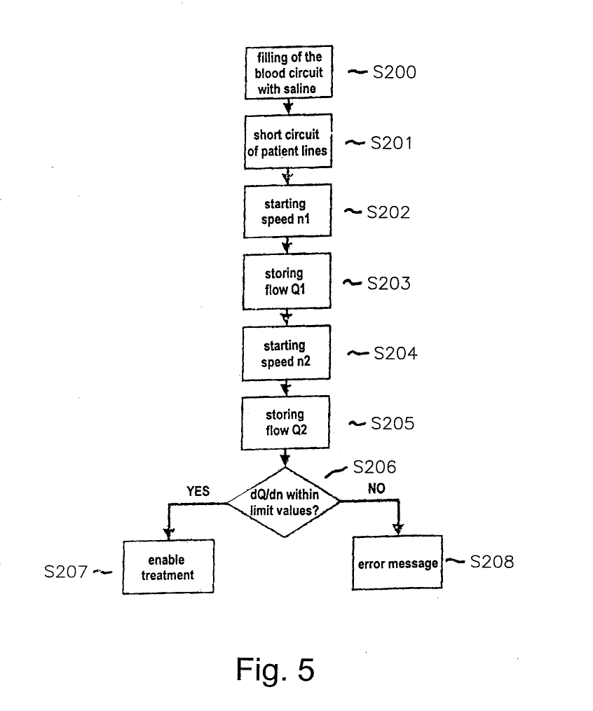

FIG. 5: shows a further flow diagram for putting the blood treatment device according to the invention into operation.

FIG. 1 shows a schematic representation of an extracorporeal blood circuit 10 of a blood treatment device according to the invention with a centrifugal pump 20, dialyzer 30, flow sensor 50 and safety clamps 45 and 85.

The extracorporeal blood circuit 10 is part of a blood treatment device and is driven by the centrifugal pump 20. Downstream of the centrifugal pump 20 a dialyzer 30 is arranged, wherein the dialyzer 30 can be or preferably is a hollow fiber membrane dialyzer 30.

The patient P is connected to the extracorporeal blood circuit 10 via a non-illustrated catheter or needles, wherein the arterial port 40 is provided with a clamp 45, so that the arterial port 40 can be disconnected. Between the clamp 45 and the centrifugal pump 20 a flow sensor 50 is provided. The arterial part of the extracorporeal blood circuit 10 is designated with the reference numeral 47.

The venous part 60 of the extracorporeal blood circuit 10 comprises the return line 62 which leads to the venous port 80 with which the treated blood is returned to the patient P. Before the venous port 80 a clamp 85 is provided, by means of which this port can be disconnected.

The present invention in particular is based on the finding that the differential pressure, which is generated by a centrifugal pump such as the centrifugal pump 20, is approximately proportional to the square of the speed of the centrifugal pump:

Furthermore, at constant viscosity and constant flow resistance the flow rate is approximately proportional to the square root of the differential pressure:

Q˜\sqrt{Δp} (Equation 2)

Hence it follows that at constant resistance and constant viscosity the flow rate is proportional to the speed of the centrifugal pump:

Q˜n (Equation 3)

with

p=pressure,

Q=flow rate,

n=speed.

The slope of this relation is dependent on the flow resistance of the circuit, as can be taken in particular from FIG. 2, which shows a diagram concerning the flow rate in dependence on the speed at different flow resistances and constant viscosity.

The viscosity of the fluid results in an offset, as can furthermore be taken from FIG. 3. By determining the flow increase at variable speed, a measure for the flow resistance of the circuit thus can be determined.

During a dialysis treatment, both changes in the viscosity, for example by ultrafiltration etc., and changes in the flow resistance occur due to kinking, clotting, needle suction etc. Both changes have a direct influence on the resulting flow rate.

Since the flow rate is an important parameter of the dialysis treatment, the same should correspond to the preset value on average. This is achieved by a control by means of a flow sensor. Changes in viscosity and flow resistance, however, lead to the fact that the controller must change the speed of the centrifugal pump. During the control operation, the flow changes caused thereby are utilized to determine the slope dQ/dn, which is a measure for the flow resistance.

Hence, it is known whether the change has been caused by the viscosity of the fluid or by the flow resistance of the circuit. The pressure measurement in the extracorporeal circuit is not able to do this, since changes both in resistance and in viscosity lead to a change in pressure.

In addition, there is always known a current measure for the flow resistance. This measure can be utilized to on the one hand initiate countermeasures and on the other hand represent an easily comprehensible, non-technical indicator for the flow resistance for example on a user interface, e.g. a display of the blood treatment device, e.g. in the form of a battery filling level indication or the like.

This determination of the differential pressure can be used as follows for example in the blood treatment device shown in FIG. 1:

For example, before each treatment, in particular a dialysis treatment, a test of the disposable articles used can be made. Before each treatment, the extracorporeal circuits of dialysis machines generally are filled with sodium chloride solutions. In short-circuited patient lines the speed is varied and the resulting flow is measured. The measure for the flow resistance dQ/dn must lie within a certain range, when the circuit is free from faults. Otherwise, the presence of faulty components is detected. With this test, the flow sensor of the extracorporeal blood circuit of the dialysis machine at the same time can be tested in functional terms.

Furthermore, a continuous monitoring of the flow resistance can be performed during the treatment. The flow rate on average is adjusted to a preset value by means of a controller. When correcting disturbing quantities such as flow resistance or viscosity, the flow change also is observed beside the change in speed, in order to therefrom derive the measure for the flow resistance dQ/dn.

Relatively fast processes such as kinking, clotting or the suction of a catheter or the arterial needle lead to the fact that the flow rate initially decreases and subsequently is corrected again. Slow processes, such as the change in viscosity due to ultrafiltration, are corrected continuously. As a further safety measure, a reversible speed change can be performed, which either is caused in a timed manner or upon exceedance of a certain speed at the flow rate determined. An example for this process is shown in FIG. 4. FIG. 4 shows a flow diagram of a reversible speed change for determining the flow resistance.

In step S100, an adjustment of Q_{blood} to Qₛₑₜ is effected.

In step S101, it is checked whether a certain time period T has elapsed. If this is not the case yet, the process returns to step S100. However, if a certain time period T has elapsed already, the process continues with step S102, which consists in storing the values for n1 and Q1.

In step S103, the speed then is adjusted by Δn.

In step S104, n2 and Q2 then are stored. In step S105, ΔQ/Δn can then be calculated.

In step S106, it then is checked whether the calculated value ΔQ/Δn lies within an allowed range. If this is the case, the process goes back to step S100. If this is not the case, a corresponding countermeasure or corresponding countermeasures is/are taken in step S107.

The detection of a flow resistance increased too much can be utilized to initiate art automatic return of blood, which for example can be a countermeasure or part of step S107. A further countermeasure according to step S107 also can consist in interrupting the treatment in the interest of the patient safety or to start a different kind of safe condition.

In addition, it is possible to provide for an easily comprehensible representation of the flow resistance for operators and patients of the blood treatment device.

Since the value dQ/dn is a measure for the flow resistance of the blood circuit or extracorporeal blood circuit 10, this value can also be utilized to provide an easily comprehensible and non-technical representation of the flow resistance for the operator of the machine (e.g. in the style of a battery filling level indication).

The value of dQ/dn, which is utilized during the initial test of the disposable article, can also be used to standardize the value. Thus, 100% would correspond to the initial value of the slope, whereas 0% is a value at which the delivery of blood through the extracorporeal circuit 10 no longer can be carried out without a greatly increased blood damage.

In addition, it is possible to detect a location of the increase in flow resistance, i.e. the point at which the increase of the flow resistance is caused.

An additional pressure sensor can be mounted for example in the extracorporeal blood circuit 10, such as between the centrifugal pump 20 and a dialyzer 30. Hence it becomes possible to localize an increased flow resistance. If the measured pressure drops, the increased flow resistance is located between patient P and pump 20. If the measured pressure increases, the increased flow resistance is located behind the pump 20, e.g. in the dialyzer 30. As a result of such localization, it becomes possible to selectively initiate countermeasures.

In the case of a catheter or needle sucked in, for example the speed of the blood pump 20 and hence the suction pressure is greatly throttled. Since the pump 20 is non-occluding, the high negative pressure does not remain between vessel, wall and catheter or needle, but is decreased. Subsequently, pressure or, in a timed manner, the speed of the centrifugal pump 20 can be increased again and the treatment can be continued. If the constriction remains, for example due to kinking of the patient hose, the safe condition must be started, i.e. the treatment must be stopped and an alarm must be issued.

Furthermore, it is possible to determine the viscosity. As can be taken from FIGS. 1 and 2, it is possible to determine the viscosity of the fluid from the speed of the pump 20 and the flow rate of the system. If the relation of speed and flow rate is described approximately with

Q≈a*n+b (Equation 4)

a corresponds to the slope dQ/dn or a parameter dependent on the flow resistance, and b corresponds to the offset or a parameter dependent on the viscosity. When the flow resistance parameter a has been determined as described, b is calculated from the current/averaged flow and the current/averaged speed to obtain:

$${b}_{current} \approx {Q}_{current} - \frac{Δ Q}{Δ n} {n}_{current} \qquad ( Equation 5 )$$

FIG. 5 shows a flow diagram concerning the initial tests when putting the extracorporeal blood circuit 10 into operation. In step S200, the filling of the extracorporeal blood circuit 10 with a sodium chloride solution is carried out. In step S201, the patient lines then are short-circuited. In step S202, the speed of the centrifugal pump 20 as shown in FIG. 1 then is started with a speed n1. In step S203, the flow rate Q1 then is stored.

In step S204, the speed then is increased to n2 and the extracorporeal blood circuit 10 is operated therewith. In step S205, the flow rate Q2 furthermore is stored

In step S206 the value dQ/dn thereupon is calculated and it is checked whether the same lies within specified limit values. If this is the case, the treatment is enabled in step S207, and if this is not the case, an error message correspondingly is issued in step S208.

1. A blood treatment device with at least one extracorporeal blood circuit (10) with a filter element (30) and with at least one centrifugal pump means (20), wherein furthermore a monitoring means is provided, by means of which the flow resistance of the extracorporeal circuit (10) can be determined, wherein the flow resistance can at least partly be determined with reference to the flow (Q) in at least one portion of the extracorporeal circuit and the speed (n) of the centrifugal pump means.

2. The blood treatment device according to claim 1, characterized in that in a first step the flow (Q11) in the at least one portion of the extracorporeal blood circuit (10) can be determined at a first speed (n1) by means of the monitoring means and in a second step the flow (Q2) in the at least one portion of the extracorporeal blood circuit (10) can be determined at a second speed (n2), wherein by means of these values (Q1, Q2; n1, n2) as a measure for the flow resistance (dQ/dn) the ratio of the differential values can be calculated.

3. The blood treatment device according to claim 2, characterized in that by means of the monitoring means a faulty condition of the extracorporeal blood circuit (10) can be detected when the measure for the flow resistance (dQ/dn) leaves a predefined range and/or exceeds or falls below a predefined threshold value.

4. The blood treatment device according to claim 3, characterized in that by means of the monitoring means at least one countermeasure can be initiated upon detection of a faulty condition.

5. The blood treatment device according to claim 4, characterized in that the countermeasure comprises the triggering of an alarm and/or the changing of the speed of the centrifugal pump means and/or the transfer of the blood treatment device into a safe condition.

6. The blood treatment device according to claim 2, characterized in that at least one display means is provided, by means of which the measure for the flow resistance (dQ/dn) can be represented and/or displayed.

7. The blood treatment device according to claim 1, characterized in that in the extracorporeal blood circuit (10) at least one pressure detection means, in particular at least one pressure sensor is provided, by means of which the pressure in at least one portion of the extracorporeal blood circuit can be determined.

8. The blood treatment device according to claim 7, characterized in that by means of the monitoring means a point in the extracorporeal blood circuit with an increased flow resistance can be determined with reference to the pressure determined by means of the pressure detection means.

9. The blood treatment device according to claim 1, characterized in that the extracorporeal blood circuit (10) is at least partly formed by a disposable cassette.

10. The blood treatment device according to claim 9 characterized in that the cassette includes at least one hose portion and/or at least one drip chamber and/or at least one valve and/or at least one filter and/or the filter element (30).

11. A monitoring means for a blood treatment device according to claim 1 with the monitoring means features according to claim 1.

12. A method for operating a blood treatment device, in particular a blood treatment device according to claim 1, comprising at least one extracorporeal blood circuit (10) with a filter element (30) and with at least one centrifugal pump means (20), wherein furthermore a monitoring means is provided, by means of which the flow resistance of the extracorporeal circuit (10) is determined, wherein the flow resistance is at least partly determined with reference to the flow (Q) in at least one portion of the extracorporeal blood circuit and the speed (n) of the centrifugal pump means.

13. The method according to claim 12, characterized in that in a first step the flow (Q1) in the at least one portion of the extracorporeal blood circuit (10) is determined at a first speed (n1) by means of the monitoring means and in a second step the flow (Q2) in the at least one portion of the extracorporeal blood circuit (10) is determined at a second speed (n2), wherein by means of these values (Q1, Q2; n1, n2) as a measure for the flow resistance (dQ/dn) the ratio of the differential values is calculated.

14. The method according to claim 13, characterized in that by means of the monitoring means a faulty condition of the extracorporeal blood circuit (10) is detected, when the measure for the flow resistance (dQ/dn) leaves a predefined range and/or exceeds or falls below a predefined threshold value.

15. The method according to claim 14, characterized in that by means of the monitoring means at least one countermeasure is initiated upon detection of a faulty condition.

16. The blood treatment device according to claim 15, characterized in that the countermeasure comprises the triggering of an alarm and/or the changing of the speed of the centrifugal pump means and/or the transfer of the blood treatment device into a safe condition.

17. The method according to claim 12, characterized in that by means of the monitoring means a point in the extracorporeal blood circuit with an increased flow resistance can be determined with reference to a determined pressure in at least one portion of the extracorporeal blood circuit or is determined in the presence of an increased flow resistance.
